(19) United States
(12) Patent Application Publication
(10) Pub. No.: US 2025/0017687 A1
(43) Pub. Date: Jan. 16, 2025

(54) MIXED REALITY SURGICAL HELMET

(71) Applicant: Zimmer, Inc., Warsaw, IN (US)

(72) Inventors: Ramnada Chav, Laval (CA); Pierre Antoine Vidal, Warsaw, IN (US)

(21) Appl. No.: 18/765,970

(22) Filed: Jul. 8, 2024

Related U.S. Application Data
(60) Provisional application No. 63/526,347, filed on Jul. 12, 2023.

(51) Int. Cl. A61B 90/00; A61B 90/35

(52) U.S. Cl. CPC A61B 90/37; A61B 90/35; A61B 2090/365

(57) ABSTRACT

A surgical helmet assembly securable to a XR device can include a frame, a surgical hood, a cooling system, and a controller. The frame can be securable to the XR device. The surgical hood can be connected to the frame and can be configured to cover at least a portion of a head of a user. The cooling system can be connected to the frame and can be locatable at least partially within the surgical hood. The cooling system can be operable to deliver an air stream to the frame, the XR device, and the surgical hood. The controller can be connected to the frame and in communication with the XR device and the cooling system. The controller can be configured to: receive a cooling system adjustment command from the user, and adjust the cooling system based on the cooling system adjustment command.

### CLAIM OF PRIORITY

This application claims the benefit of U.S. Provisional Patent Application Ser. No. 63/526,347, filed on Jul. 12, 2023, the benefit of priority of which is claimed hereby, and which is incorporated by reference herein in its entirety.

### BACKGROUND

Surgical advancements have allowed surgeons to use preoperative planning, display devices within a surgical field, optical imaging, and guides to improve surgical outcomes and customize surgery for a patient. While these advances have allowed for quicker and more successful surgeries, they rely on physical objects, which have costs and time requirements for manufacturing and configuration. Physical objects and devices can also obstruct portions of a surgical field.

Computer-assisted surgery is a growing field that encompasses a wide range of devices, uses, procedures, and computing techniques, such as surgical navigation, pre-operative planning, and various robotic techniques. In computer-assisted surgery procedures, a navigation system, such as an augmented reality, mixed reality, or other system can be used in some surgical procedures, such as orthopedic procedures, to aid a surgeon in completing the procedures more accurately, more quickly, or with less fatigue.

### BRIEF DESCRIPTION OF THE DRAWINGS

In the drawings, which are not necessarily drawn to scale, like numerals can describe similar components in different views. Like numerals having different letter suffixes can represent different instances of similar components. The drawings illustrate generally, by way of example, but not by way of limitation, various embodiments discussed in the present document.

FIG. 1 illustrates a surgical field.

FIG. 2 illustrates a perspective view of a portion of a surgical helmet assembly.

FIG. 3 illustrates a perspective view of a portion of a surgical helmet assembly.

FIG. 4 illustrates a perspective view of a portion of a surgical helmet assembly.

FIG. 5 illustrates a schematic view of a surgical helmet assembly.

FIG. 6 illustrates a perspective view of a surgical field including a mixed reality display.

FIG. 7 illustrates a perspective view of a surgical field including a mixed reality display.

FIG. 8 illustrates a perspective view of a surgical field including a mixed reality display.

FIG. 9 illustrates a perspective view of a surgical field including a mixed reality display.

FIG. 10 illustrates a method of operating a surgical helmet assembly.

FIG. 11 illustrates a block diagram illustrating an example of a machine upon which one or more embodiments may be implemented.

### DETAILED DESCRIPTION

Mixed reality devices can be used during surgeries to help improve surgical performance or to help surgeons or physicians interact with devices during the surgery. These devices can be worn by physicians and at least the lenses of the devices must be exposed to the environment to operate properly. However, the components of the surgical helmet can be difficult to control during a surgical procedure while maintaining the sterile field.

This disclosure helps to address these issues by providing a surgical helmet assembly that includes a frame securable to an XR device (which can be an augmented reality device, virtual reality device, or mixed reality device) and a surgical hood connected to the frame and configured to cover at least a portion of a head of a user. The surgical helmet assembly can also include a cooling system connected to the frame and locatable at least partially within the surgical hood. The cooling system can be operable to deliver an air stream to the frame, the XR device, and the surgical hood. The surgical helmet assembly or the XR device can also include a controller connected to the frame and in communication with the XR device and the cooling system, where the controller is configured to receive a cooling system adjustment command from the user and adjust the cooling system based on the cooling system adjustment command. The controller can be configured to receive other system adjustment commands from the user and adjust the surgical helmet assembly based on the commands, where the commands can be vocal commands, air touch commands, head gaze commands, eye gaze commands, or the like. Adjustment of settings of the surgical helmet assembly, such as a temperature within the hood, based on the commands can allow the surgeon or other professional to control the surgical helmet assembly during a procedure quickly, easily, and without disrupting sterility of the surgical field.

The above discussion is intended to provide an overview of subject matter of the present patent application. It is not intended to provide an exclusive or exhaustive explanation of the invention. The description below is included to provide further information about the present patent application.

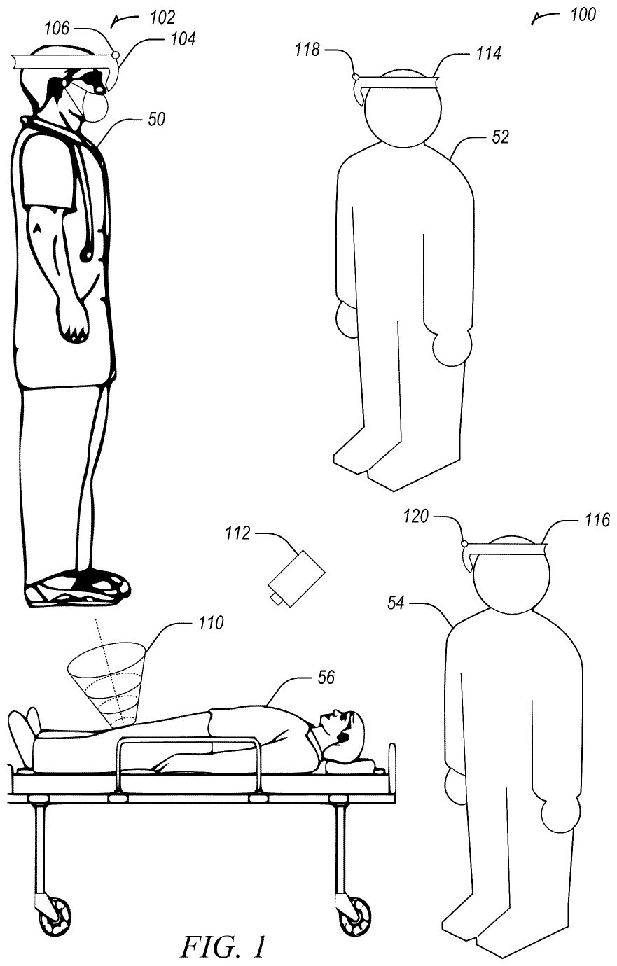

FIG. 1 illustrates a surgical field 100 in accordance with some embodiments. The surgical field 100 illustrated in FIG. 1 can include a surgeon 50, a patient 56, and can include a camera 112. The surgeon 50 can wear a surgical helmet assembly 102 (examples of which are discussed below in further detail) that can include augmented reality (AR), virtual reality (VR), or mixed reality (MR) device 104 (e.g., XR device 104, which can be any type of these devices or head mounted display (HMD)), which can be used to display a virtual object 110 to the surgeon 50. The virtual object 110 may not be visible to others within the surgical field 100 (e.g., surgical assistant 52 or nurse 54), though they may wear an ZR devices 114 and 116 respectively (which can include cameras 118 and 120, respectively). Even if another person is viewing the surgical field 100 with an XR device, the person may not be able to see the virtual object 110 or may be able to see the virtual object 110 in a shared augmented reality with the surgeon 50, or may be able to see a modified version of the virtual object 110 (e.g., according to customizations unique to the surgeon 50 or the person) or may see different virtual objects entirely. Augmented reality is explained in more detail below.

Augmented reality is a technology for displaying virtual or “augmented” objects or visual effects overlaid on a real environment. Mixed reality is a similar technology where visual effects are anchored or located relative to real objects in an environment. The disclosure herein can apply to either technology in addition to virtual reality, or the like

The real environment can include a room or specific area (e.g., the surgical field 100), or can be more general to include the world at large. The virtual aspects overlaid on the real environment can be represented as anchored or in a set position relative to one or more aspects of the real environment. For example, the virtual object 110 can be configured to appear to be resting on a table. An XR system can present virtual aspects that are fixed to a real object without regard to a perspective of a viewer or viewers of the system (e.g., the surgeon 50). For example, the virtual object 110 can exist in a room, visible to a viewer of the system within the room and not visible to a viewer of the system outside the room. The virtual object 110 in the room can be displayed to the viewer outside the room when the viewer enters the room. In this example, the room can act as a real object that the virtual object 110 can be fixed to in the system.

The XR device 104 can include one or more screens or displays, such as a single screen or two screens (e.g., one per eye of a user). The screens can be holographic displays, or the like configured to display or project images. The screens can allow light to pass through the screens such that aspects of the real environment are visible to the surgeon 50 while displaying the virtual object 110. The virtual object 110 can be made visible to the surgeon 50 by projecting light. The virtual object 110 can appear to have a degree of transparency or can be opaque (i.e., blocking aspects of the real environment).

Virtual objects can be a computer generated 3D representation of an object that can be displayed on 2D screens (computer screens, augmented reality (embedded screen) or virtual reality (stereo screen). Holographic objects can include 3D projection(s) of an object (not necessarily on a screen) formed through the interference of light wave(s) to create a realistic 3D image that can be viewed from different angle and immerged within the real world. The objects referenced herein can be virtual objects or holographic objects.

An XR system can be viewable to one or more viewers, and can include differences among views available for the one or more viewers while retaining some aspects as universal among the views. For example, a heads-up display can change between two views while virtual objects can be fixed to a real object or area in both views. Aspects such as a color of an object, lighting, or other changes can be made among the views without changing a fixed position of at least one virtual object.

In an example, the user can interact with the virtual object 110, such as by moving the virtual object 110 from a first position to a second position. For example, the user can move an object with his or her hand. This can be done in the XR system virtually by determining that the hand has moved into a position coincident or adjacent to the object (e.g., using one or more cameras, which can be mounted on an XR device, such as XR device camera 106 or separate, and which can be static or can be controlled to move), and causing the object to move in response. Virtual aspects can include virtual representations of real world objects or can include visual effects, such as lighting effects, etc. The XR system can include rules to govern the behavior of virtual objects, such as subjecting a virtual object to gravity or friction, or can include other predefined rules that defy real world physical constraints (e.g., floating objects, perpetual motion, etc.). An XR device 104 can include a camera 106 (not to be confused with the camera 112, separate from the XR device 104). The XR device camera 106 or the camera 112 can include an infrared camera, an infrared filter, a visible light filter, a plurality of cameras, a depth camera, or the like. The XR device 104 can project virtual items over a representation of a real environment, which can be viewed by a user.

The surgeon 50 can interact with the XR device 104 in several ways such as eye tracking (eye gaze), head gaze, air touch, vocal commands, or the like. Eye tracking can be or can include determining a focal point of the eye(s) of the surgeon 50, such as by using input from one or more sensors of the XR device 104. Head gaze can be or can include determining a location of a field of view (or virtual field of view) of the surgeon 50 using one or more sensors of the XR device 104. Air touch can be or can include determining a location or gesture of one or more body part (e.g., finger(s) or hand) of the surgeon 50. Vocal commands can be or can include determining a command based on detected audio (e.g., talking or making other noises) from the surgeon 50.

The XR device 104 can be used in the surgical field 100 during a surgical procedure, for example performed by the surgeon 50 on the patient 56. The XR device 104 can project or display virtual objects, such as the virtual object 110 during the surgical procedure to augment the surgeon's vision. The surgeon 50 can control the virtual object 110 using the XR device 104, a remote controller for the XR device 104, or by interacting with the virtual object 110 (e.g., using a hand to “interact” with the virtual object 110 or a gesture recognized by the camera 106 of the XR device 104). The virtual object 110 can augment a surgical tool. For example, the virtual object 110 can appear (to the surgeon 50 viewing the virtual object 110 through the XR device 104) as a representation of a landmark previously placed on a patient bone. In another example, the virtual object 110 can be used to represent a planned location of a landmark (e.g., using a pre-operative image and a captured image of the bone in the real space). In certain examples, the virtual object 110 can react to movements of other virtual or real-world objects in the surgical field. For example, the virtual object 110 can be altered to move a landmark (e.g., a placed landmark). In other examples, the virtual object 110 can be a virtual representation of a remote surgical field (e.g., an entire OR, a camera field of view of a room, a close-up view of a surgical theater, etc.). In this example, the virtual object 110 can include a plurality of virtual objects.

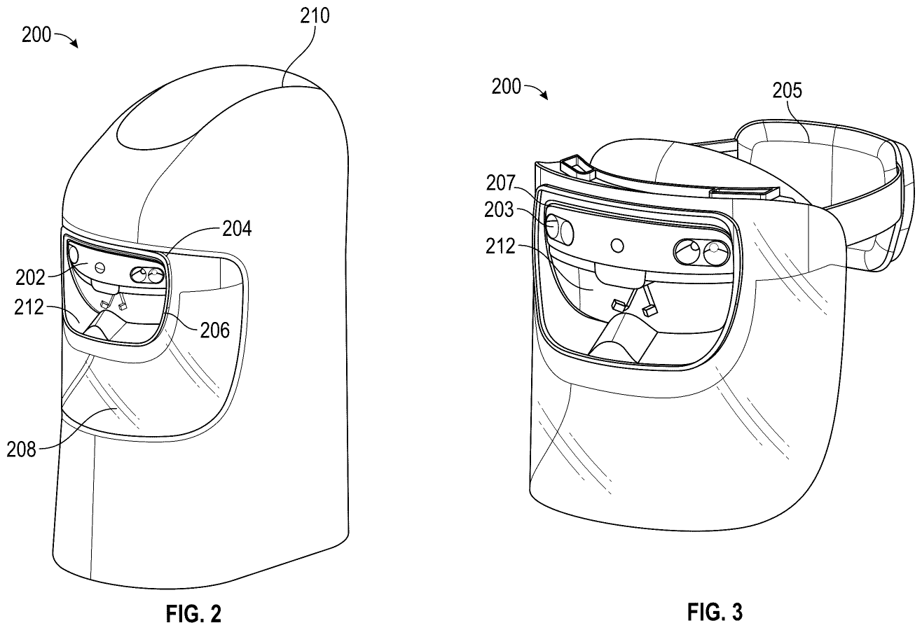

FIG. 2 illustrates an isometric view of a surgical helmet assembly 200. FIG. 3 illustrates an isometric view of a portion of the surgical helmet assembly 200. FIGS. 2 and 3 are discussed together below. The surgical helmet assembly 200 can include an XR device 202, which can be similar to those discussed above and can include a sensor array 203, a battery pack 205, and a body 207.

The body 207 can support the lenses 212 and other components of the XR device 202. An inner frame 204 can be a polymer member releasably securable to one or more components of the XR device 202, such as the body 207. An outer frame 206 can include a visor 208 and a hood 210 and the outer frame 206 can be releasably securable to the inner frame 204, such as through one or more of hook and loop fasteners, clamps, clips, temporary adhesive, or the like. The hood 210 can be configured to at least partially surround a head of the surgeon and the visor 208 can be oriented in front of a face of the user to help increase peripheral visibility. The hood 210 can be secured to the visor 208 and the hood 210 and the visor 208 can be disposable or sterilizable following removal from the inner frame 204.

The sensors 203 can include one or more visual or optical sensors such as a camera, LiDAR, fiber optic, infrared sensor, or the like. The sensor array 203 can also include or one or more other sensors such as an audio sensor, electromagnetic sensor, or the like. The sensors 203 can be connected to the controller of the XR device 202 or a controller of the surgical helmet assembly 200 and can be configured to transmit signals to one or more of the controllers. The battery pack 205 can be connected to a rear portion of one or more of the inner frame 204 or the outer frame 206 and can be configured to provide power to one or more components of the surgical helmet assembly 200 or the XR device 202.

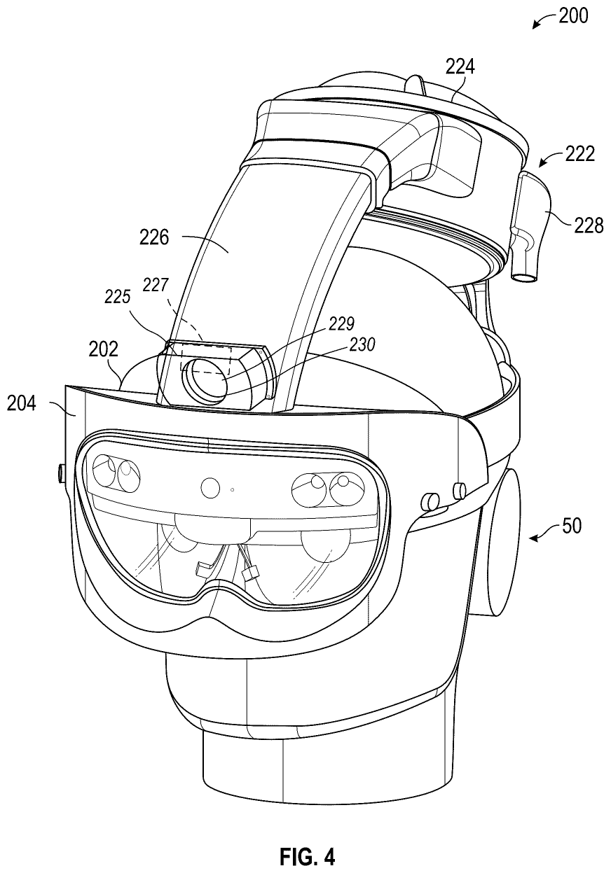

FIG. 4 illustrates an isometric view of a portion of a surgical helmet assembly 200. The surgical helmet assembly 200 can be similar to the surgical helmet assemblies discussed above. The surgical helmet assembly 200 can include a cooling system. Any of the surgical helmet assemblies discussed above or below can include such a system.

The surgical helmet assembly 200 can include a cooling system 222 connected to an inner frame 204 and connectable to the XR device 202. The cooling system 222 can generally be locatable or located within the surgical hood. Generally, the cooling system 222 can be operable to deliver conditioned air to the inner frame 204, the XR device 202, and the surgical hood.

Optionally, the cooling system 222 can include a fan or blower 224 connected to a duct or plenum 226 (downstream) and openings 228 (upstream). The blower 224 can be operated to draw air through the openings 228 and the blower 224 to discharge air through the plenum 226 and to the inner frame 204 and the XR device 202, such as through the XR device 202, to help cool the user 50 and to help limit fogging of the visor and lenses of the XR device 202. Optionally, the air stream can be reversed such that air is drawn through the frames 204 and 206 and the XR device 202 and is drawn through the plenum 226 to the blower 224 and is discharged through the openings 228. The cooling system 222 can optionally include other cooling components, such as an active cooling or heating system (e.g., refrigeration, chilled water, thermoelectric (Peltier) cooler, or the like) that can optionally include one or more heat exchangers.

The surgical helmet assembly 200 can also include a light assembly 225 that can be secured to the inner frame 204 or the outer frame 206 (or the cooling system 222) of the XR device 202. The light assembly 225 can include an actuator 227 that can be in communication with a controller of the surgical helmet assembly 200 or the XR device 202 such that the controller can be operable to adjust an angle of the light assembly 225 to adjust a direction of light emitted from the surgical helmet assembly 200. The light assembly 225 can also include a lens 229 that can include one or more actuators that can be in communication with a controller of the surgical helmet assembly 200 or the XR device 202 such that the controller can be operable to adjust a beam pattern of the light assembly 225. Optionally, the light assembly 225 can be configured to adjust an intensity of the light. Optionally, the light assembly 225 can also include an adjustable aperture 230 that can be in communication with the controller(s) for adjusting the beam pattern. Optionally, the controller can adjust a color of the light (e.g., cool vs. warm when using light emitting diodes, though other combinations or red, yellow, and blue can be used).

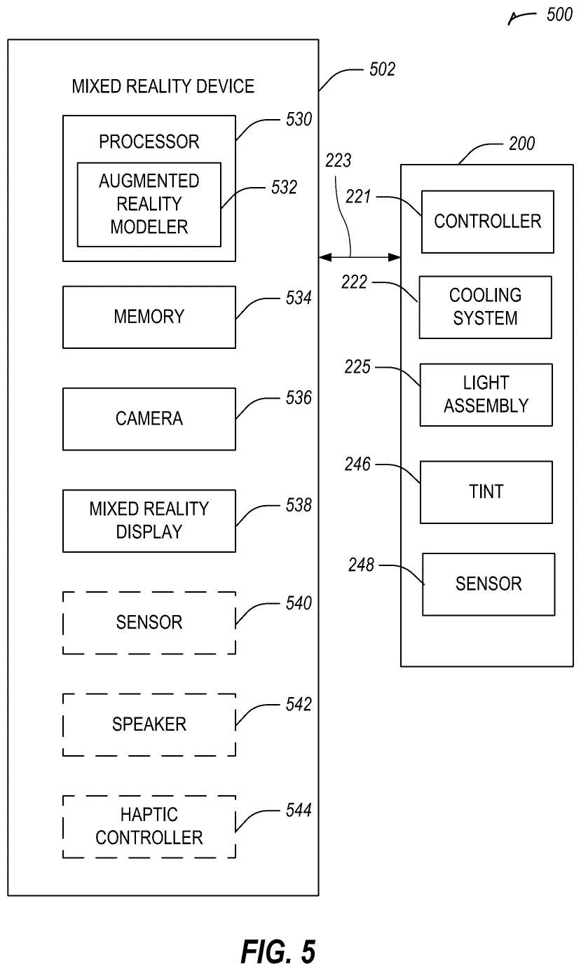

FIG. 5 illustrates a system 500 for surgical instrument identification using an augmented reality display in accordance with some embodiments. The system 500 can be used to perform any of the techniques described above or below, for example, by using a processor 530. The system 500 includes a XR device 502 that can be in communication with the surgical helmet assembly 200, which can include a controller 221 or can use the processor 530. The surgical helmet assembly 200 and the XR device 502 can be connected through an interface 223, such as a cable connecting the XR device 502 to the surgical helmet assembly 200. Optionally, the interface 223 can be at least partially wireless such that the XR device 502 and the surgical helmet assembly 200 can communicate through one or more wireless communication protocols, such as those listed below.

The XR device 502 can include a processor 530, memory 534, an XR display 538, and a camera 536. The XR device 502 can include a sensor 540, a speaker 542, or a haptic controller 544. The surgical helmet assembly 200 can include cooling system 222.

The processor 530 of the XR device 502 can include a XR modeler 532 (which can be an MR, AR, VR, or XR modeler). The XR modeler 532 can be used by the processor 530 to create the augmented reality environment. For example, the XR modeler 532 can receive dimensions of a room, such as from the camera 536 or sensor 540, and can create the augmented reality environment to fit within the physical structure of the room. In another example, physical objects can be present in the room and the XR modeler 532 can use the physical objects to present virtual objects in the augmented reality environment. For example, the XR modeler 532 can use or detect a table present in the room and present a virtual object as resting on the table. The XR display 538 can display the XR environment overlaid on a real environment. The display 538 may show a virtual object, using the XR device 502, such as in a fixed position in the XR environment or a floating position (e.g., via head tracking). The XR modeler 532 can receive a video stream of a remote surgical field for virtually displaying within the room. In an example, a dimension of a virtual object (e.g., a remote surgical field) can be modified (e.g., shrunk) to be virtually displayed within the room. The XR device 502 can provide a zoom function to allow a user to zoom in on a portion of a virtual object (e.g., within a virtual surgical field). Such a process or system using a HMD camera equipped with analog or digital zoom to magnify features such as variable zoom factor, auto focus, auto tracking, stabilizer, image correction, or the like can allow for elimination of a surgical loupe that are commonly worn on top of lenses

The XR device 502 can include a sensor 540, such as an infrared sensor. The camera 536 or the sensor 540 can be used to detect movements, such as a gesture by a surgeon or other user, that can be interpreted by the processor 530 as attempted or intended interaction by the user with the virtual target. The processor 530 can identify an object in a real environment, such as through processing information received using the camera 536.

The XR display 538, for example during a surgical procedure, can present, such as within a surgical field while permitting the surgical field to be viewed through the mixed reality display, a virtual feature corresponding to a physical feature hidden by an anatomical aspect of a patient. The virtual feature can have a virtual position or orientation corresponding to a first physical position or orientation of the physical feature. In an example, the virtual position or orientation of the virtual feature can include an offset from the first physical position or orientation of the physical feature. The offset can include a predetermined distance from the augmented reality display, a relative distance from the augmented reality display to the anatomical aspect, or the like.

FIG. 5 also shows that the system 500 can include a controller 221, the cooling system 222, the light assembly 225, a tint mechanism 246 and a sensor 248. The tint mechanism 246 can be connected to the lenses 212 or the visor 208 and can be operable (such as via the controller 221 or the processor 530) to adjust a tint of the lenses 212 or the visor 208, such as based on a command from the user (e.g., the surgeon 50). For example, an electrochromic tint (or the like) can be adjustable.

The sensor 248 can be one or more sensors connected to the surgical helmet assembly 200, such as to the inner frame 204 or the outer frame 206. The sensor 248 can be in communication with the controller 221 such as to transmit a signal thereto. In one example, the sensor 248 can be a temperature sensor configured to generate a temperature signal based on a temperature within the hood 210 of the surgical helmet assembly 200.

Optionally, the processor 530 or the controller 221 can disabled certain types of commands, e.g., air touch, during certain portions of a surgical procedure. For example, head gaze or eye gaze can be disabled during performing of a surgical step, such as reaming or drilling of a bone to help avoid accidentally selecting a virtual object during important steps of the surgical procedure.

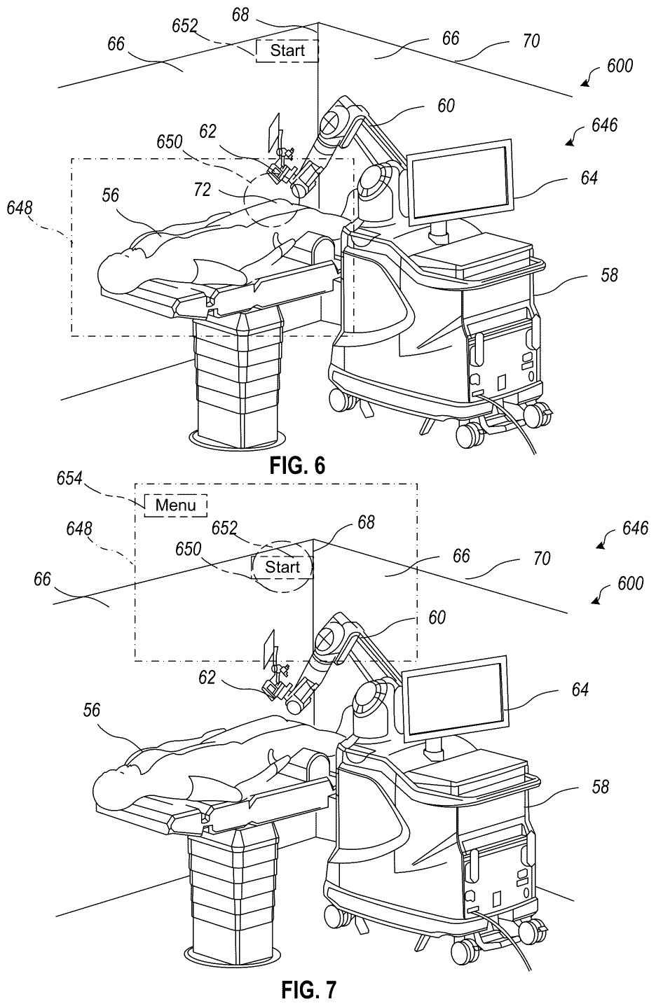

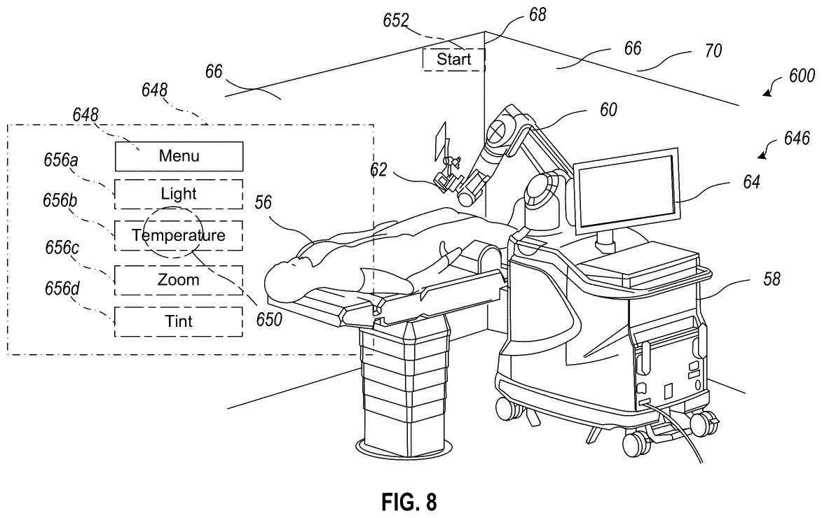

FIG. 6 illustrates a perspective view of a surgical field 600 including a XR display 646 including a virtual field of view 648. FIG. 7 illustrates a perspective view of the surgical field 600 including the XR display 646. FIG. 8 illustrates a perspective view of the surgical field 600 including the XR display 646. FIGS. 6-8 are discussed together below and illustrate how one or more of the XR devices and surgical helmet assemblies discussed above or below can be used.

For example, FIGS. 6 and 7 show a surgical field 600 that can include portions of a surgical environment, such as the patient 56, a surgical robot 58 (including a robotic arm 60, an end effector 62, and a display 64). The surgical field 600 can also include walls 66 (meeting at a corner 68) and a ceiling 70. FIGS. 6-8 also show that the virtual field of view 648 that can be displayed or projected by the XR device 502 (or the XR device 202), such as onto a visor of the surgical helmet assembly 200 or the lenses 212. The virtual field of view 648 can be overlayed on the surgical field 600 based on a position or orientation of a head or face of the user.

The virtual field of view 648 can include a virtual focal zone 650 that can be determined by the one or more sensors of the XR device 202 via eye tracking, head gaze, or the like. For example, as eyes of the surgeon 50 are tracked, the virtual focal zone 650 can be displayed or overlayed on the surgical field 600 based on a determined focal region of eyes of the surgeon 50 wearing the surgical helmet assembly 200 and XR device 202. For example, the virtual focal zone 650 can be displayed or overlayed on a surgical site 72 (e.g., a knee of the patient 56) when the surgeon 50 focuses on the surgical site 72, as shown in FIG. 6.

FIGS. 6-8 also show a start indication 652, which can be a virtual object projected by the XR device (e.g., the XR device 202 or the XR device 502) that can be overlayed or projected onto the surgical field 600. The start indication 652 can be located near or at the corner 68, such as near or at the ceiling 70 of the surgical field 600. The location of the start indication 652 can be located by the surgeon 50, such as during preoperative setup or can be moved at any time by the surgeon 50 using a command (e.g., vocal, air touch, or the like). The location of the start indication 652 can be purposely away from the patient 56 and the surgical site 72 so that the start indication 652 is not accidentally selected during a procedure.

As shown in FIG. 6, the start indication 652 can be located out of the virtual field of view 648. As shown in FIG. 7, the surgeon 50 can move the virtual field of view 648 to include the start indication 652, allowing the surgeon 50 to select the start indication 652 such as by moving the virtual focal zone 650 onto or over the start indication 652, such as via eye tracking or eye gaze processing by the XR device 502. For example, when the virtual focal zone 650 is positioned by the surgeon 50 over the start indication 652 the XR device 202 or the surgical helmet assembly 200 can determine that the start indication 652 is selected.

Optionally, the surgeon 50 can select the start indication 652 using head gaze, such as by moving a portion of the virtual field of view 648 over the start indication 652 (e.g., a center of the virtual field of view 648, a corner of the virtual field of view 648, or the like), such as by moving the head to select the start indication 652. For example, when the surgeon 50 positions a center of the virtual field of view 648 over the start indication 652, the XR device 202 or the surgical helmet assembly 200 can determine that the start indication 652 is selected. Optionally, the surgeon 50 can select the start indication 652 using air touch such as by using one or more gestures when the start indication 652 is in the virtual field of view 648.

Optionally, the surgeon 50 can select the start indication 652 using vocal commands. Optionally, the start indication 652 can be inactive or not visible and can be accessed using voice or audio command(s). For example, the surgeon 50 can state “hey, toga” or other similar utterances or statements to activate the start indication 652. Also, optionally, the start indication can be bypassed or not used and audio or voice commands can be used to adjust other settings. For example, the surgeon 50 can state one or more of “hey, TOGA, set the temperature to 68,” “hey, TOGA, change the light intensity to 7,” or “hey, TOGA, enable magnifying glass to 10×.”

As shown in FIG. 7, once the start indication 652 is selected via one or more methods, the XR device 202 or the surgical helmet assembly 200 can generate a virtual menu indication 654 that can be selectable to produce a menu command. For example, the virtual menu indication 654 can be selected via eye gaze (or focus), head gaze, air touch, or voice commands to generate a menu command, such that when the menu command is received, a virtual menu can be presented within the surgical field 600 by the XR device 202 or the surgical helmet assembly 200.

For example, as shown in FIG. 8, when the virtual menu indication 654 of FIG. 7 is selected, the virtual menu indication 654 can be moved or relocated or can be optionally kept in place. The virtual menu indication 654 can also change shapes, sizes, or colors following selection. The virtual menu indication 654 can include one or more system adjustment indications 656 selectable to produce a system adjustment command within the surgical field, using an XR display of the XR device 202 or the surgical helmet assembly 200, while permitting the surgical field 600 to be viewed through the XR display (e.g., the lenses 212). In some examples, following selection of the virtual menu indication 654, the virtual menu indication 654 and the system adjustment indications 656 can be pinned to the virtual field of view 648 such that the virtual menu indication 654 and the system adjustment indications 656 move with the virtual field of view 648.

The one or more system adjustment indications 656 can include a light indication 656a, a cooling system indication 656b, a zoom indication 656c, and a tint indication 656d. The one or more system adjustment indications 656 can include other settings, such as temperature, humidity, light, or the like. The one or more system adjustment indications 656 can include other indications selectable to adjust other system settings, such as location of a critical zone, location of one or more indications, or other settings. The light indication 656a can be an indication selectable to produce one or more menus or indications to control one or more settings of a light of the surgical helmet assembly 200 (e.g., the light assembly 225). For example, the light indication 656a can be selectable to produce a menu to adjust light intensity, an angle of the light, or a focus (or beam pattern) of the light assembly 225.

The cooling system indication 656b can be an indication selectable to produce one or more menus or indications to control one or more settings of the cooling system 222. For example, the cooling system indication 656b can be selectable to produce a menu to adjust a speed of the blower 224, a temperature within the hood 210, or the like. The zoom indication 656c can be an indication selectable to produce one or more menus or indications to control one or more settings of the display of the surgical helmet assembly 200, such as a virtual zoom window, as discussed in further detail below. The tint indication 656d can be an indication selectable to produce one or more menus or indications to control one or more settings of the lenses 212 or the visor 208, such as a type of tint or an amount of tint.

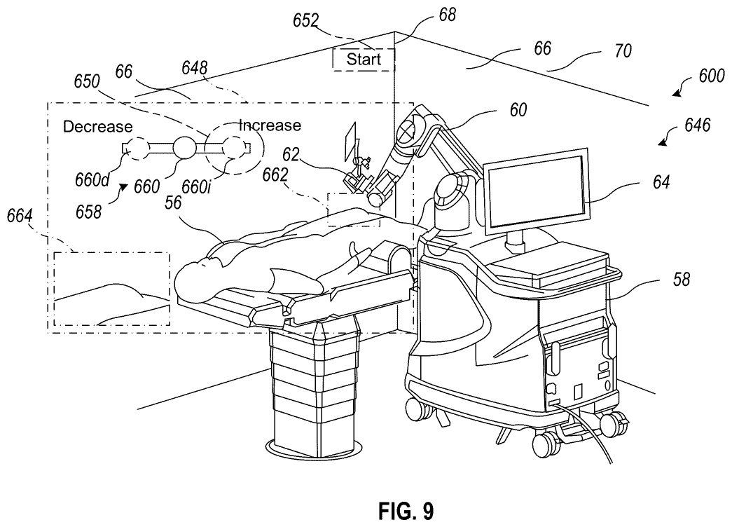

FIG. 9 illustrates a perspective view of the surgical field 600 including a XR display 646 including the virtual field of view 648. The surgical field 600 and XR display 646 of FIG. 9 can be consistent with FIGS. 6-8 discussed above. FIG. 9 shows additional actions or interactions that can be performed using the surgical helmet assembly 200 or the XR device 202. For example, FIG. 9 shows that when one of the system adjustment indications 656 are selected, an adjustment indication 658 can be displayed or overlayed onto the surgical field 600, such as within the XR display 646 and optionally within the view 648, such as depending on a head position or orientation of the patient 56. That is, the adjustment indication 658 can be in a fixed location relative to the surgical field 600 or can be in a fixed location relative to the view 648.

The adjustment indication 658 can include a slider indication 660 that can be adjustable to increase or decrease the selected setting, such as using air touch, eye gaze, head gaze, or voice commands. The slider indication 660 can be operated to adjust an angle of the light assembly 225, an intensity of the light assembly 225, or a focus or beam pattern of the light assembly 225 when the light indication 656a is previously selected. Optionally, the surgeon 50 can select the light indication 656a to maintain focus of the light emitted by the light assembly 225 on a specific area, such as the surgical site 72. For example, the controller 221 or the processor 530 can operate the actuator 227 to maintain the light emitted onto the surgical site 72 as the surgeon 50 moves.

The slider indication 660 can be operated by the user to adjust tint of the visor 208 or the lenses 212 when the tint indication 656d is previously selected. The slider indication 660 can be operated to adjust zoom of a virtual zoom window when the zoom indication 656c is previously selected, and the slider indication 660 can be operated to adjust the cooling system 222 when the tint indication 656d is previously selected.

For example, the virtual focal zone 650 can be used to adjust the slider indication 660 to the position 660i to increase the setting, as shown in FIG. 9. When the slider indication 660 is adjusted, a system adjustment command can be generated and the processor 530 or the controller 221 can receive the system adjustment command and the processor 530 or the controller 221 can adjust an accessory of the surgical helmet assembly 200 based on the system adjustment command. For example, when the cooling system indication 656b is previously selected, the surgeon 50 can adjust the slider indication 660 to the position 660i to increase the blower speed. Optionally, when the cooling system indication 656b is previously selected, the surgeon 50 can adjust the slider indication 660 to the position 660d to reduce a temperature within the hood 210.

FIG. 9 also shows a zoom indication 662 and a zoom window 664. When the zoom indication 656c is previously selected, the slider indication 660 can be adjusted to increase or decrease an amount that the area viewable in the zoom indication 662 is enlarged within the zoom window 664. For example, the slider indication 660 can be adjusted to increase zoom between a multiple of 1 (1×) and 100×, between 1× and 50×, between 1× and 25×, between 1× and 10×, between 1× and 5×, or the like. Optionally, when the zoom indication 656c is selected, the zoom indication 662 or the zoom window 664 can be movable or positionable (e.g., within the surgical field 600, the XR display 646, or the virtual field of view 648) by the surgeon 50, such as by using air touch, voice commands, eye gaze, head gaze, other gestures, or the like.

The indication 662 can also be or can separately (or simultaneously) be a critical zone indication 662. The critical zone indication 662 can be a box, area, or portion of the XR display 646 or virtual field of view 648 that is an area that needs to be viewed at all times, such as all of, or a portion of the surgical site 72. As such, the XR device 202 or the surgical helmet assembly 200 can limit or prevent one or more of the indications from being positioned over the critical zone indication 662, even when an item that is pinned to the virtual field of view 648 moves across the critical zone indication 662. For example, when the start indication 652 is pinned or locked with respect to the virtual field of view 648, the surgical helmet assembly 200 or the XR device 202 can move or alter a location of the start indication 652 with respect to the virtual field of view 648 to avoid the start indication 652 from passing through the critical zone 662.

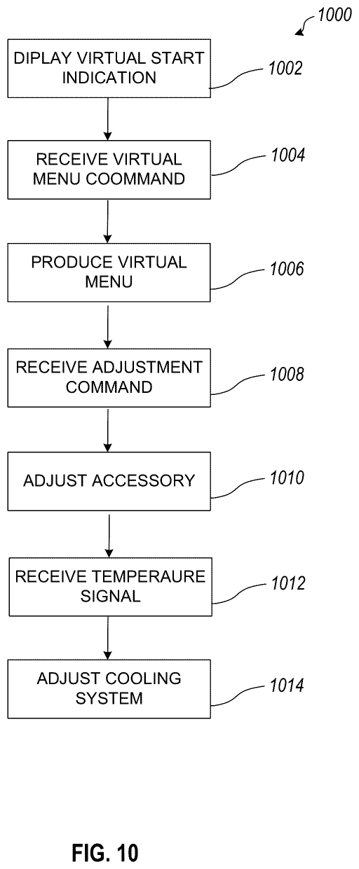

FIG. 10 illustrates a method 1000. The method 1000 can be a method of operating a surgical helmet assembly. More specific examples of the method 1000 are discussed below. The steps or operations of the method 1000 are illustrated in a particular order for convenience and clarity; many of the discussed operations can be performed in a different sequence or in parallel without materially impacting other operations. The method 1000 as discussed includes operations performed by multiple different actors, devices, or systems. It is understood that subsets of the operations discussed in the method 1000 can be attributable to a single actor, device, or system could be considered a separate standalone process or method.

The method 1000 can begin at step 1002 where a virtual start indication selectable to produce the virtual menu indication can be presented within the surgical field, using the mixed reality display of the XR device. For example, the start indication 652 can be presented within the surgical field 600 using the lenses 212 (or a display thereof) of the XR device 202. At step 1004, a menu command can be received at a processor of the surgical helmet assembly (e.g., the surgical helmet assembly 200) or the XR device (e.g., the XR device 202).

At step 1006, when the menu command is received, a virtual menu indication including one or more system adjustment indications selectable to produce a system adjustment command can be produced within the surgical field, using a mixed reality display of the XR device, while permitting the surgical field to be viewed through the XR display. For example, the XR device 202 can produce the virtual menu indication 654 which can include the one or more system adjustment indications 656 that can be selectable to produce a system adjustment command. At step 1008, the system adjustment command can be received at the processor (e.g., the processor 530 or the controller 221). At step 1010, an accessory of the surgical helmet assembly can be adjusted based on the system adjustment command. For example, the light assembly 225 or the cooling system 222 can be adjusted based on the system adjustment command.

Optionally, at step 1012, a temperature signal indicative of a temperature within a surgical hood of the surgical helmet assembly can be received. For example, a temperature signal from the sensor 248 can be received (e.g., by the controller 221). At step 1014 a cooling system of the surgical helmet assembly can be adjusted based on the system adjustment command and the temperature signal. For example, the controller 221 can adjust the cooling system 222 of the surgical helmet assembly 200 based on the system adjustment command and the temperature signal, such as to increase or decrease a fan speed.

In some examples, as the XR device or surgical helmet assembly move relative to the surgical field, a location of the virtual start indication 652 can be maintained or fixed relative to the surgical field. In some examples a critical zone of the surgical field can be defined and a location of the virtual start indication or the virtual menu indication can be altered relative to the XR display, such as to prevent presenting the virtual start indication 652 or the virtual menu indication 654 within the critical zone (e.g., the critical zone 662).

In some examples, the system adjustment command can include an air touch command, an eye gaze command, or a head gaze command. In some examples, the cooling system can include a cooling fan connected to the frame and operable to generate an air stream to flow through the surgical hood, wherein the processor is configured to increase or decrease a speed of the cooling fan based on the system adjustment command and based on the temperature signal. In some examples, the cooling system includes a light assembly connected to at least one of the frame, the surgical hood, or the XR device, wherein the processor is configured to control an intensity of the light assembly based on the system adjustment command. In some examples, the virtual start indication is projected, by the XR display, onto a portion of a user wearing the surgical helmet assembly. In some examples, the virtual menu indication is presented during only a first portion of a surgical procedure, and wherein the virtual menu indication is not produced during a second portion of the surgical procedure.

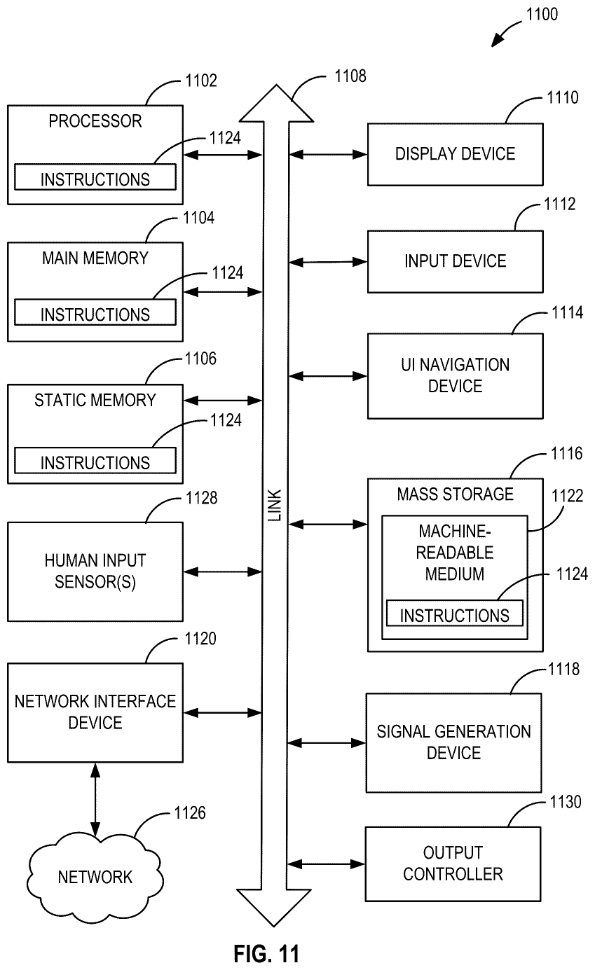

FIG. 11 illustrates a block diagram of an example machine 1100 upon which any one or more of the techniques (e.g., methodologies) discussed herein may perform. Examples, as described herein, may include, or may operate by, logic or a number of components, or mechanisms in the machine 1100. Circuitry (e.g., processing circuitry) is a collection of circuits implemented in tangible entities of the machine 1100 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 1100 follow.

In alternative embodiments, the machine 1100 may operate as a standalone device or may be connected (e.g., networked) to other machines. In a networked deployment, the machine 1100 may operate in the capacity of a server machine, a client machine, or both in server-client network environments. In an example, the machine 1100 may act as a peer machine in peer-to-peer (P2P) (or other distributed) network environment. The machine 1100 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (Saas), other computer cluster configurations.

The machine (e.g., computer system) 1100 may include a hardware processor 1102 (e.g., a central processing unit (CPU), a graphics processing unit (GPU), a hardware processor core, or any combination thereof), a main memory 1104, a static memory (e.g., memory or storage for firmware, microcode, a basic-input-output (BIOS), unified extensible firmware interface 1106 (UEFI), etc.), and mass storage 1108 (e.g., hard drive, tape drive, flash storage, or other block devices) some or all of which may communicate with each other via an interlink (e.g., bus) 1130. The machine 1100 may further include a display unit 1110, an alphanumeric input device 1112 (e.g., a keyboard), and a user interface (UI) navigation device 1114 (e.g., a mouse). In an example, the display unit 1110, input device 1112 and UI navigation device 1114 may be a touch screen display. The machine 1100 may additionally include a storage device 1108 (e.g., drive unit), a signal generation device 1118 (e.g., a speaker), a network interface device 1120, and one or more sensors 1116, such as a global positioning system (GPS) sensor, compass, accelerometer, or other sensor. The machine 1100 may include an output controller 1128, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the processor 1102, the main memory 1104, the static memory 1106, or the mass storage 1108 may be, or include, a machine readable medium 1122 on which is stored one or more sets of data structures or instructions 1124 (e.g., software) embodying or utilized by any one or more of the techniques or functions described herein. The instructions 1124 may also reside, completely or at least partially, within any of registers of the processor 1102, the main memory 1104, the static memory 1106, or the mass storage 1108 during execution thereof by the machine 1100. In an example, one or any combination of the hardware processor 1102, the main memory 1104, the static memory 1106, or the mass storage 1108 may constitute the machine readable media 1122. While the machine readable medium 1122 is illustrated as a single medium, the term “machine readable medium” may include a single medium or multiple media (e.g., a centralized or distributed database, and/or associated caches and servers) configured to store the one or more instructions 1124.

The term “machine readable medium” may include any medium that is capable of storing, encoding, or carrying instructions for execution by the machine 1100 and that cause the machine 1100 to perform any one or more of the techniques of the present disclosure, or that is capable of storing, encoding or carrying data structures used by or associated with such instructions. Non-limiting machine readable medium examples may include solid-state memories, optical media, magnetic media, and signals (e.g., radio frequency signals, other photon based signals, sound signals, etc.). In an example, a non-transitory machine readable medium comprises a machine readable medium with a plurality of particles having invariant (e.g., rest) mass, and thus are compositions of matter. Accordingly, non-transitory machine-readable media are machine readable media that do not include transitory propagating signals. Specific examples of non-transitory machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 1124 may be further transmitted or received over a communications network 1126 using a transmission medium via the network interface device 1120 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engincers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 1120 may include one or more physical jacks (e.g., Ethernet, coaxial, or phone jacks) or one or more antennas to connect to the communications network 1126. In an example, the network interface device 1120 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 1100, and includes digital or analog communications signals or other intangible medium to facilitate communication of such software. A transmission medium is a machine readable medium.

### Notes and Examples

The following, non-limiting examples, detail certain aspects of the present subject matter to solve the challenges and provide the benefits discussed herein, among others.

Example 1 is a surgical helmet assembly securable to a XR device, the surgical helmet assembly comprising: a frame securable to the XR device; a surgical hood connected to the frame and configured to cover at least a portion of a head of a user; a cooling system connected to the frame and locatable at least partially within the surgical hood, the cooling system operable to deliver an air stream to the frame, the XR device, and the surgical hood; and a controller connected to the frame and in communication with the XR device and the cooling system, the controller configured to: receive a cooling system adjustment command from the user; and adjust the cooling system based on the cooling system adjustment command.

In Example 2, the subject matter of Example 1 includes, wherein the cooling system adjustment command is a voice command, an air touch command, an eye gaze command, or a head gaze command.

In Example 3, the subject matter of Examples 1-2 includes, a temperature sensor configured to produce a temperature signal based on a temperature within the surgical hood, the controller configured to adjust the cooling system based on the cooling system adjustment command and the temperature signal.

In Example 4, the subject matter of Example 3 includes, wherein the cooling system includes a cooling fan connected to the frame and operable to generate the air stream, wherein the controller is configured to increase or decrease a speed of the cooling fan based on the cooling system adjustment command and based on the temperature signal.

In Example 5, the subject matter of Example 4 includes, wherein the controller is configured to present, within a surgical field, using a mixed reality display of the XR device, while permitting the surgical field to be viewed through the mixed reality display, a virtual slider indication that is user selectable to variably adjust the speed of the cooling fan.

In Example 6, the subject matter of Example 5 includes, wherein the virtual slider indication is an air touch slider.

Example 7 is a surgical helmet assembly securable to a XR device, the surgical helmet assembly comprising: a frame securable to the XR device; a surgical hood connected to the frame and configured to cover at least a portion of a head of a user, the surgical hood including a visor; a light assembly connected to at least one of the frame, the surgical hood, or the XR device, the light assembly configured to emit light through the visor and toward a surgical field; and a controller connected to the frame and in communication with the XR device and the light assembly, the controller configured to: receive a light adjustment command from the user; and adjust the light based on the light adjustment command.

In Example 8, the subject matter of Example 7 includes, wherein the light adjustment command is a voice command, an air touch command, an eye gaze command, or a head gaze command.

In Example 9, the subject matter of Examples 7-8 includes, wherein the controller is configured to adjust a direction of the light, a beam pattern of the light, or an intensity of the light based on the light adjustment command.

In Example 10, the subject matter of Example 9 includes, wherein the controller is configured to present, within a surgical field, using a mixed reality display of the XR device, while permitting the surgical field to be viewed through the mixed reality display, a virtual slider indication that is user selectable to variably adjust the intensity of the light assembly.

Example 11 is a method for using, in a surgical field, a surgical helmet assembly and XR device connectable to a frame of the surgical helmet assembly, the method comprising: receiving, at a processor of the surgical helmet assembly or the XR device, a menu command; presenting, when the menu command is received, within the surgical field, using a mixed reality display of the XR device, while permitting the surgical field to be viewed through the mixed reality display, a virtual menu indication including one or more system adjustment indications selectable to produce a system adjustment command; receiving, at the processor, the system adjustment command; and adjusting an accessory of the surgical helmet assembly based on the system adjustment command.

In Example 12, the subject matter of Example 11 includes, wherein the system adjustment command includes an air touch command, an eye gaze command, or a head gaze command.

In Example 13, the subject matter of Examples 11-12 includes, receiving a temperature signal indicative of a temperature within a surgical hood of the surgical helmet assembly; and adjusting a cooling system of the surgical helmet assembly based on the system adjustment command and the temperature signal.

In Example 14, the subject matter of Example 13 includes, wherein the cooling system includes a cooling fan connected to the frame and operable to generate an air stream to flow through the surgical hood, wherein the processor is configured to increase or decrease a speed of the cooling fan based on the system adjustment command and based on the temperature signal.

In Example 15, the subject matter of Examples 13-14 includes, wherein the cooling system includes a light assembly connected to at least one of the frame, the surgical hood, or the XR device, wherein the processor is configured to control an intensity of the light assembly based on the system adjustment command.

In Example 16, the subject matter of Examples 11-15 includes, presenting, within the surgical field, using the mixed reality display of the XR device, a virtual start indication selectable to produce the virtual menu indication.

In Example 17, the subject matter of Example 16 includes, maintaining, as the XR device or surgical helmet assembly move relative to the surgical field, a location of the virtual start indication relative to the surgical field.

In Example 18, the subject matter of Examples 16-17 includes, wherein the virtual start indication is projected, by the mixed reality display, onto a portion of a user wearing the surgical helmet assembly.

In Example 19, the subject matter of Examples 16-18 includes, wherein the virtual menu indication is presented during only a first portion of a surgical procedure, and wherein the virtual menu indication is not produced during a second portion of the surgical procedure.

In Example 20, the subject matter of Examples 16-19 includes, defining a critical zone of the surgical field; and altering a location of the virtual start indication or the virtual menu indication relative to the mixed reality display to prevent presenting the virtual start indication or the virtual menu indication within the critical zone.

Example 21 is a non-transitory machine-readable medium including instructions, for controlling, in a surgical field, a surgical helmet assembly and XR device connectable to a frame of the surgical helmet assembly, which when executed by a machine, cause the machine to: receive, at a processor of the surgical helmet assembly or the XR device, a menu command; present, when the menu command is received, within the surgical field, using a mixed reality display of the XR device, while permitting the surgical field to be viewed through the mixed reality display, a virtual menu indication including one or more system adjustment indications selectable to produce a system adjustment command; receive, at the processor, the system adjustment command; and adjust an accessory of the surgical helmet assembly based on the system adjustment command.

In Example 22, the subject matter of Example 21 includes, wherein the system adjustment command includes an air touch command, an eye gaze command, or a head gaze command.

In Example 23, the subject matter of Examples 21-22 includes, the instructions to further cause the machine to: receive a temperature signal indicative of a temperature within a surgical hood of the surgical helmet assembly; and adjust a cooling system of the surgical helmet assembly based on the system adjustment command and the temperature signal.

In Example 24, the subject matter of Example 23 includes, wherein the cooling system includes a cooling fan connected to the frame and operable to generate an air stream to flow through the surgical hood, wherein the processor is configured to increase or decrease a speed of the cooling fan based on the system adjustment command and based on the temperature signal.

In Example 25, the subject matter of Examples 23-24 includes, wherein the cooling system includes a light assembly connected to at least one of the frame, the surgical hood, or the XR device, wherein the processor is configured to control an intensity of the light assembly based on the system adjustment command.

In Example 26, the subject matter of Examples 21-25 includes, the instructions to further cause the machine to: present, within the surgical field, using the mixed reality display of the XR device, a virtual start indication selectable to produce the virtual menu indication.

In Example 27, the subject matter of Example 26 includes, the instructions to further cause the machine to: maintain, as the XR device or surgical helmet assembly move relative to the surgical field, a location of the virtual start indication relative to the surgical field.

In Example 28, the subject matter of Examples 26-27 includes, wherein the virtual start indication is projected, by the mixed reality display, onto a portion of a user wearing the surgical helmet assembly.

In Example 29, the subject matter of Examples 26-28 includes, wherein the virtual menu indication is presented during only a first portion of a surgical procedure, and wherein the virtual menu indication is not produced during a second portion of the surgical procedure.

In Example 30, the subject matter of Examples 26-29 includes, the instructions to further cause the machine to: define a critical zone of the surgical field; and alter a location of the virtual start indication or the virtual menu indication relative to the mixed reality display to prevent presenting the virtual start indication or the virtual menu indication within the critical zone.

Example 31 is at least one machine-readable medium including instructions that, when executed by processing circuitry, cause the processing circuitry to perform operations to implement of any of Examples 1-30.

Example 32 is an apparatus comprising means to implement of any of Examples 1-30.

Example 33 is a system to implement of any of Examples 1-30.

Example 34 is a method to implement of any of Examples 1-30.

In Example 35, the apparatuses, systems, or methods of any one or any combination of Examples 1-34 can optionally be configured such that all elements or options recited are available to use or select from.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) can be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A surgical helmet assembly securable to a XR device, the surgical helmet assembly comprising:
a frame securable to the XR device;
a surgical hood connected to the frame and configured to cover at least a portion of a head of a user;
a cooling system connected to the frame and locatable at least partially within the surgical hood, the cooling system operable to deliver an air stream to the frame, the XR device, and the surgical hood; and
a controller connected to the frame and in communication with the XR device and the cooling system, the controller configured to:
receive a cooling system adjustment command from the user; and
adjust the cooling system based on the cooling system adjustment command.

2. The surgical helmet assembly of claim 1, wherein the cooling system adjustment command is a voice command, an air touch command, an eye gaze command, or a head gaze command.

3. The surgical helmet assembly of claim 1, comprising:
a temperature sensor configured to produce a temperature signal based on a temperature within the surgical hood, the controller configured to adjust the cooling system based on the cooling system adjustment command and the temperature signal.

4. The surgical helmet assembly of claim 3, wherein the cooling system includes a cooling fan connected to the frame and operable to generate the air stream, wherein the controller is configured to increase or decrease a speed of the cooling fan based on the cooling system adjustment command and based on the temperature signal.

5. The surgical helmet assembly of claim 4, wherein the controller is configured to present, within a surgical field, using a mixed reality display of the XR device, while permitting the surgical field to be viewed through the mixed reality display, a virtual slider indication that is user selectable to variably adjust the speed of the cooling fan.

6. The surgical helmet assembly of claim 5, wherein the virtual slider indication is an air touch slider.

7. A surgical helmet assembly securable to a XR device, the surgical helmet assembly comprising:
a frame securable to the XR device;
a surgical hood connected to the frame and configured to cover at least a portion of a head of a user, the surgical hood including a visor;
a light assembly connected to at least one of the frame, the surgical hood, or the XR device, the light assembly configured to emit light through the visor and toward a surgical field; and
a controller connected to the frame and in communication with the XR device and the light assembly, the controller configured to:
receive a light adjustment command from the user; and
adjust the light based on the light adjustment command.

8. The surgical helmet assembly of claim 7, wherein the light adjustment command is a voice command, an air touch command, an eye gaze command, or a head gaze command.

9. The surgical helmet assembly of claim 7, wherein the controller is configured to adjust a direction of the light, a beam pattern of the light, or an intensity of the light based on the light adjustment command.

10. The surgical helmet assembly of claim 9, wherein the controller is configured to present, within a surgical field, using a mixed reality display of the XR device, while permitting the surgical field to be viewed through the mixed reality display, a virtual slider indication that is user selectable to variably adjust the intensity of the light assembly.

11. A non-transitory machine-readable medium including instructions, for controlling, in a surgical field, a surgical helmet assembly and XR device connectable to a frame of the surgical helmet assembly, which when executed by a machine, cause the machine to:
receive, at a processor of the surgical helmet assembly or the XR device, a menu command;
present, when the menu command is received, within the surgical field, using a mixed reality display of the XR device, while permitting the surgical field to be viewed through the mixed reality display, a virtual menu indication including one or more system adjustment indications selectable to produce a system adjustment command;
receive, at the processor, the system adjustment command; and
adjust an accessory of the surgical helmet assembly based on the system adjustment command.

12. The non-transitory machine-readable medium of claim 11, wherein the system adjustment command includes an air touch command, an eye gaze command, or a head gaze command.

13. The non-transitory machine-readable medium of claim 11, the instructions to further cause the machine to:
receive a temperature signal indicative of a temperature within a surgical hood of the surgical helmet assembly; and
adjust a cooling system of the surgical helmet assembly based on the system adjustment command and the temperature signal.

14. The non-transitory machine-readable medium of claim 13, wherein the cooling system includes a cooling fan connected to the frame and operable to generate an air stream to flow through the surgical hood, wherein the processor is configured to increase or decrease a speed of the cooling fan based on the system adjustment command and based on the temperature signal.

15. The non-transitory machine-readable medium of claim 13, wherein the cooling system includes a light assembly connected to at least one of the frame, the surgical hood, or the XR device, wherein the processor is configured to control an intensity of the light assembly based on the system adjustment command.

16. The non-transitory machine-readable medium of claim 11, the instructions to further cause the machine to:
present, within the surgical field, using the mixed reality display of the XR device, a virtual start indication selectable to produce the virtual menu indication.

17. The non-transitory machine-readable medium of claim 16, the instructions to further cause the machine to:
maintain, as the XR device or surgical helmet assembly move relative to the surgical field, a location of the virtual start indication relative to the surgical field.

18. The non-transitory machine-readable medium of claim 16, wherein the virtual start indication is projected, by the mixed reality display, onto a portion of a user wearing the surgical helmet assembly.

19. The non-transitory machine-readable medium of claim 16, wherein the virtual menu indication is presented during only a first portion of a surgical procedure, and wherein the virtual menu indication is not produced during a second portion of the surgical procedure.

20. The non-transitory machine-readable medium of claim 16, the instructions to further cause the machine to:
define a critical zone of the surgical field; and
alter a location of the virtual start indication or the virtual menu indication relative to the mixed reality display to prevent presenting the virtual start indication or the virtual menu indication within the critical zone.
